(19) United States
(12) Patent Application Publication
(10) Pub. No.: US 2008/0103568 A1
(43) Pub. Date: May 1, 2008

(54) Thermal wrap arrangement for applying heat/cold to the mandibular region of the head

(76) Inventor: Rebecca A. Dow, Lake George, NY (US)

Correspondence Address: Alfred F. Hoyte, Esq., Suite 500, 1101 30th Street, N.W., Washington, DC, 20007, US

(21) Appl. No.: 12/003,495

(22) Filed: Dec. 26, 2007

Related U.S. Application Data
(63) Continuation-in-part of application No. 11/444,390, filed on Jun. 1, 2006.

(51) Int. Cl. A61F 7/00

(52) U.S. Cl. 607/109; 607/112; 607/114

(57) ABSTRACT

A thermal wrap having identical side portions, each having a pocket formed therein for receiving thermal material such as gel packs. The side portions are connected at the top by at least two independently attached straps, and at the bottom by a singe, relatively wide chin strap to ensure that the pockets allow for even application of thermal energy to the mandibular region of the face. The pockets are sized to receive standard sized gel packs.

### CROSS REFERENCE TO RELATED APPLICATION

This application is a continuation in part of application Ser. No. 11/444,390.

### BACKGROUND OF THE INVENTION

1. Field of the Invention

The present invention concerns therapeutic thermal treatment devices. More particularly, the invention is directed to a thermal treatment wrap configured for application of heat/cold to various parts of a human head.

2. Description of the Prior Art

Thermal treatment wraps and the like are well known in the art. They typically consist of a web or sheet material which may be textile, elastic, or plastic; configured or configurable to cover a particular body part. They are typically conceived as one size fits all, or, in the alternative, limited to two or three sizes. While these arrangements are generally satisfactory, they can be ineffective when used to treat specific areas of the head and face. This is primarily because the human head, unlike other body parts, changes in both size and contour from one individual to the next, and allowances have to be made for the bulk of the hair, as well as particular hairstyles. To that end, the typical therapeutic thermal head wrap has adjustable or elasticated straps, and one or more pockets or voids for containing thermal material such as gel packs, ice, etc. The drawback with these types of arrangements is that it is difficult to maintain effective thermal contact with an area of the face to be treated. This problem is exacerbated by the use of gel packs which are dense, heavy, and not particularly conforming to contoured surfaces, the result being that the gel pack will either sag or pucker, and therefore be only marginally effective at a given treatment area. Another problem with prior art devices is that the device will typically have a single large pocket for containing a gel pack, the pocket not conforming to any of the standard gel pack sizes.

U.S. Pat. No. 5,837,004 issued to Lavore is typical of the head and face thermal treatment accessories. The device has opposing pockets for containing thermal material, the pockets connected at opposite ends by a single adjustable strap. The device is one size fits all. By contrast, the present invention is directed to a thermal compress arrangement having identical side portions connected at one end by a pair of elasticated straps, and at the other end by a chin strap.

U.S. Pat. No. 5,188,103 issued to Smith discloses a simple elastic band having a repositionable thermal pouch. The present invention contemplates a thermal treatment wrap designed for treatment of the mandibular region which has pockets sized in accordance with standard gel pack sizes. The wrap is available in a range of sizes, with the pocket size selected in accordance with both the standard gel pack sizes and the size of the user's head.

The present invention is directed to a thermal wrap having identical side portions, each having a pocket formed therein for receiving thermal material such as gel packs. The side portions are connected at the top by at least two independently attached straps, and at the bottom by a singe, relatively wide chin strap to ensure that the pockets allow for even application of thermal energy to the mandibular region of the face. The pockets are sized to receive standard sized gel packs.

### SUMMARY OF THE INVENTION

It is a major object of the invention to provide an improved thermal wrap.

It is another object of the invention to provide an improved thermal wrap particularly adapted for use on a human head.

It is another object of the invention to provide an improved thermal wrap for treatment of the mandibular region of a human head.

It is another object of the invention to provide an improved, adjustable thermal wrap for treatment of the mandibular region of a human head.

It is yet another object of the invention to provide an improved thermal wrap with means for receiving standard sized thermal treatment packs.

Finally, it is a general goal of the invention to provide improved elements and arrangements thereof in an apparatus for the purposes described which is dependable and fully effective in accomplishing its intended purposes.

These and other objects of the present invention will become readily apparent upon further review of the following specification and drawings.

The present invention meets or exceeds all the above objects and goals. Upon further study of the specification and appended claims, further objects and advantages of this invention will become apparent to those skilled in the art.

### BRIEF DESCRIPTION OF THE DRAWINGS

Various other objects, features, and attendant advantages of the present invention will become more fully appreciated as the same becomes better understood when considered with the accompanying drawings, in which like reference characters designate the same or similar parts throughout the several views, and wherein:

FIG. 1 shows a front view of the thermal wrap of the invention positioned on a user.

FIG. 2 shows a cross sectional front view of the thermal wrap of FIG. 1.

FIG. 3 shows a plan view of the thermal wrap.

FIG. 4 shows a cross sectional top view of the thermal wrap.

FIG. 5 shows a front view of an alternative embodiment of the thermal wrap.

FIG. 6 shows a perspective view, partly in section, of the embodiment of FIG. 5.

### DETAILED DESCRIPTION OF THE PREFERRED EMBODIMENTS

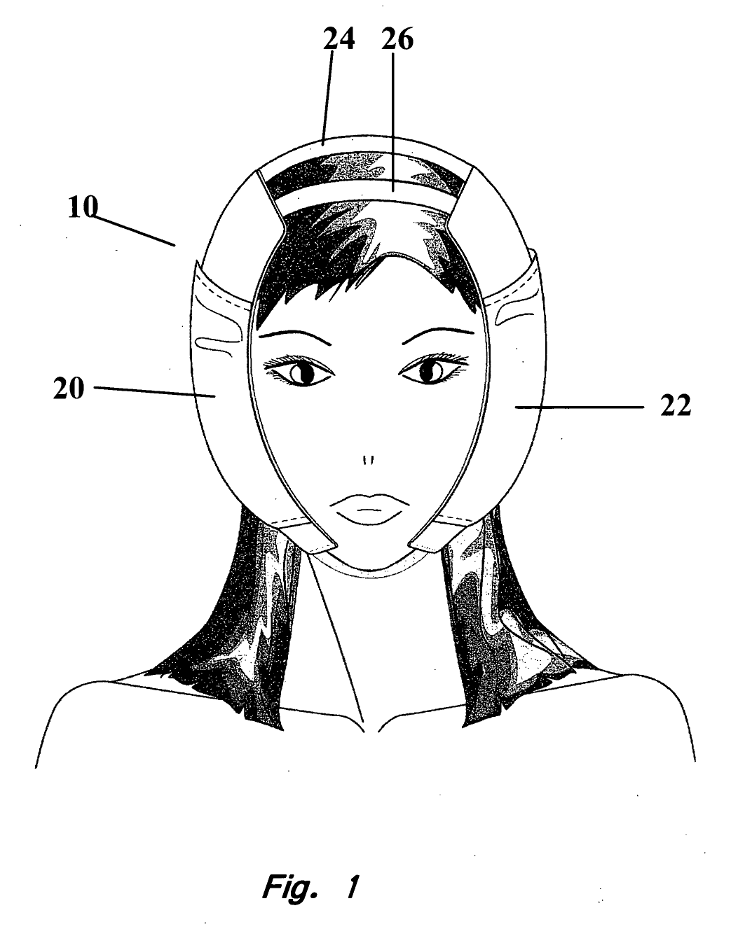

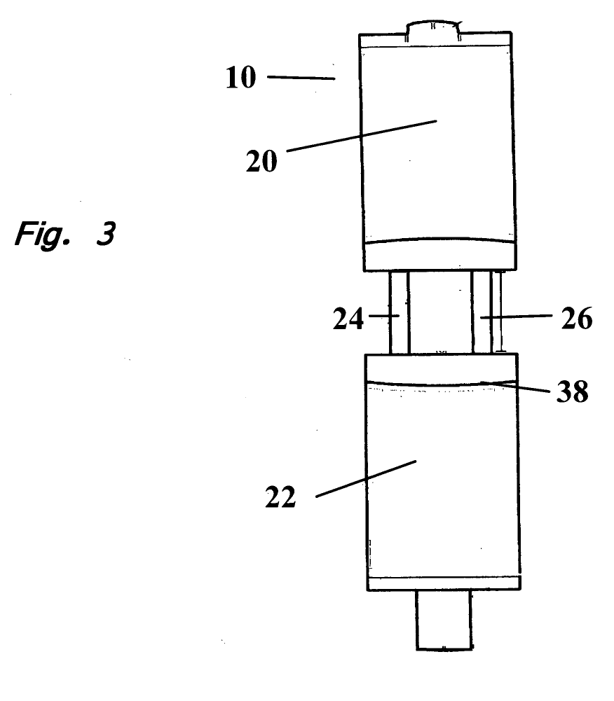

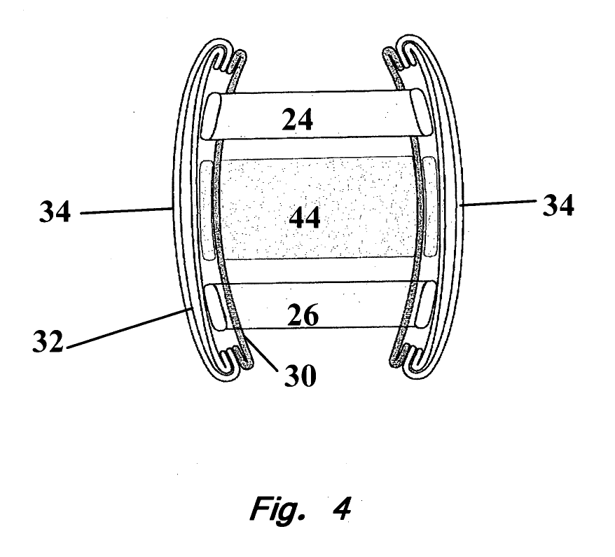

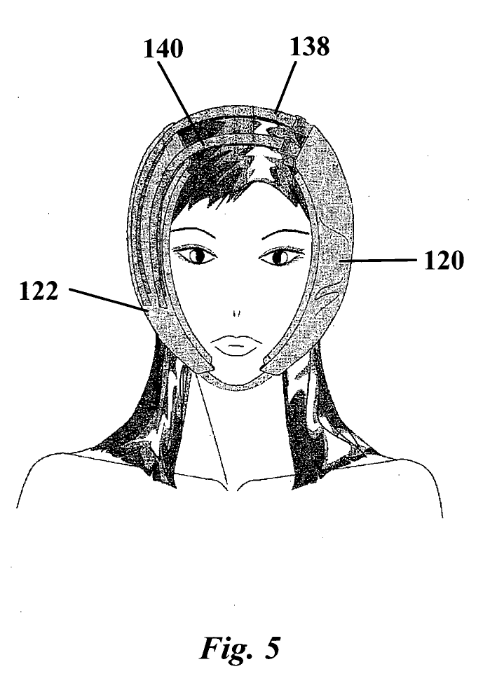

Referring now to FIGS. 1-4, the thermal wrap arrangement of the present invention, generally indicated by the numeral 10, is shown. It can be seen that the wrap 10 has a one piece main body with two nearly identical side portions 20, 22, connected by a pair of elongated elasticated straps 24, 26. The straps 24, 26 may alternatively be of a hook and loop type fastener construction, in which case they may be correspondingly longer to ensure a sufficiently large overlap area for secure fastening, and to allow for adjustment as described below in connection with an alternative embodiment. Straps 24, 26, which may extend lengthwise within the side portions, are evenly spaced about the center of the short side of opposing ends of the side portions 20, 22, and are preferably connected thereto by stitching. It should be noted that while the side portions 20, 22 are shown as rectangular, they may have any elongated shape provided the side pocket can be sized to accept standard sized gel packs as will be explained in more detail later. The wrap 10 is available in incrementally increasing sizes starting from a small head (circumference) or child's head up to a maximum of about 28 inches. The size of the side portions 20, 22 are increased in accordance with the wrap 10 size increases, which are preferably about two inch increments, the side portions 20, 22 increased by about one inch for each wrap 10 size increase, with strap 24, 26 increasing about ¾ inches. Of course the length of the straps may be increased to allow for additional adjustability. In an alternative one size fits all embodiment, the side portions 20, 22 may be an intermediate size, while the straps 24, 26 are elongated to allow for adjustment from the smallest to the largest head size. In this case, the straps 24, 26 are of a two piece configuration and may include buckles or other adjustable fastening means as shown in FIG. 5, to allow for, e.g., sliding adjustment. The even spacing of the independently attached side straps 24, 26 help reduce twisting of the wrap 10 due to the thickness of the user's hair or the way in which the hair is arranged. It can be appreciated that more than two straps 24, 26 may be employed, especially for larger sizes of the wrap, but using more than four straps would render the connection between side portions 20, 22 substantially continuous and therefore subject to twisting etc.

In a preferred embodiment, the wrap 10 is made from a polyester, spandex material, with the technical side of the material brushed or sueded to enhance user comfort. Also, the material is preferably treated with anti-microbial agents, and is woven to be quick drying. A material such as COOLMAX FRESH EFFECTS® by DUPONT® may be ideally used for this purpose. Alternatively, the wrap may be made from terrycloth, cotton or other highly absorbent material.

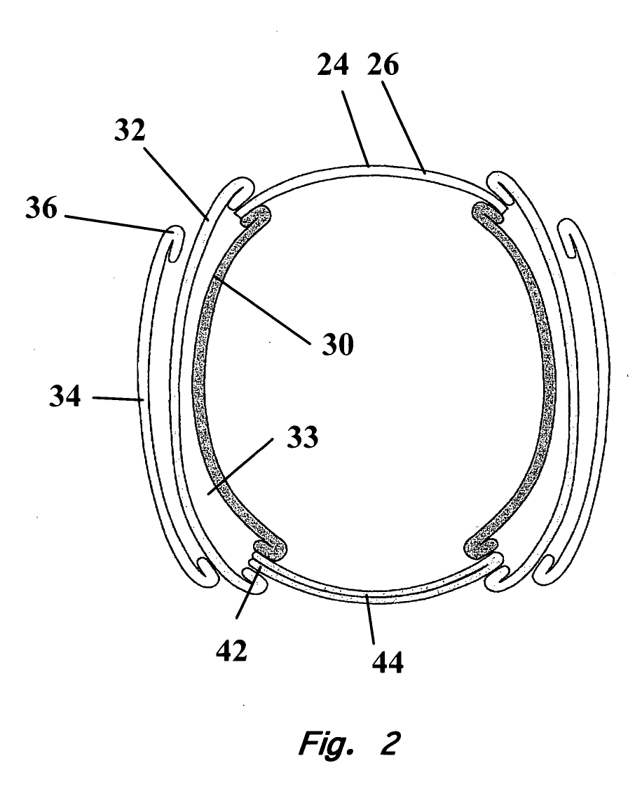

Referring specifically to FIG. 2, It can be seen that each side portion 20, 22 has both inner and outer sheets 30, 32 forming a closed envelope 33, with a third sheet 34 unattached along its top edge 36 and thereby forming a pocket 38 into which a gel pack (not shown) or the like may be inserted. The inner sheets 30 are connected to an interior facing side of each of the straps 24, 26 at their top ends, while the outer sheets 32 are connected to an exterior facing side of the traps 24, 26. The attachment of sheets 30, 32 is preferably secured by stitching, with the top and bottom edges of the inner and outer sheets 30, 32 folded over as shown to enhance durability. A key aspect of the invention is that the width of the pocket 38 is to remain constant, while the length, L, can be adjusted in accordance with the range of standard sized gel packs. As previously stated, the wrap 10 is sized in accordance with the size or circumference of a user's head, as measured from the top of the head over and around the mandibular region as can be seen in FIG. 1. Specifically, gel packs come in increments of about one inch lengthwise, and are no wider than the width of the pocket 38 which is preferably slightly less than six inches.

The bottom edges 42 of the side pocket envelope 33 are connected by a chin strap 44, in the same manner as the straps 24, 26, i.e., folded over and stitched. The chin strap 44 may also be elasticated. The chin strap 44 is relatively wide to ensure that inner and outer sheets 30, 32 are stretched taut about the mandibular region of the user's face, and is at least half the width of the side pockets 38. The sizing of the chin strap 44 relative to the width of the pockets 38 overcomes a tendency common in prior art thermal wraps where bunching or other fabric deformations due to a single, relatively thin strap, lead to an uneven application of the thermal treatment. The side pocket 38 is wide enough so that the gel pack may be slid horizontally to adjust the region to which thermal energy is transferred.

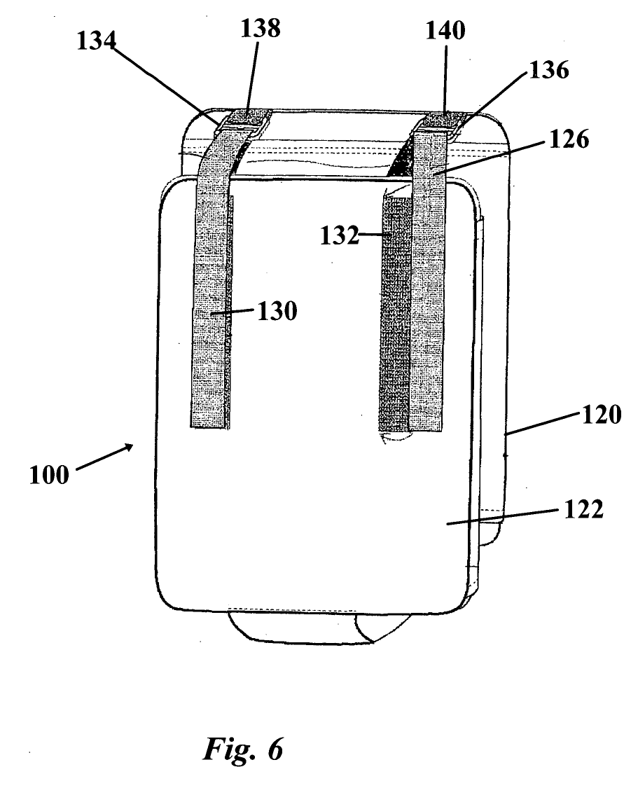

Referring now to FIGS. 5 and 6 an alternative embodiment of the invention, generally indicated by the numeral 100, allowing for additional conformance to the shape of a particular user's head, is shown. It can be seen that straps 124, 126 are elongated to extend nearly the entire length of side pocket 122. This configuration allows for straps 124, 126 to hold the pocket 122 securely to one side of the user's face. The interior side of straps 124, 126 are covered with or comprised of a hook and loop type fastener material, which can be arranged for releasable engagement with strips 130, 132 of hook and loop material, the strips being secured along their entire length to side pocket 122. Buckles 134, 136 are attached to a short length of straps 138, 140, which straps 138, 140 are attached by e.g. stitching to the top edge of pocket 120 to allow for adjustment as would be apparent to one of skill in the art. Specifically, straps 124, 126 may be looped through one side of the respective buckle 134, 136, adjusted as desired, and then secured by pressing the appropriate length of strap 124, 126 onto itself as can be seen in FIG. 6.

In use, once the user has selected a size appropriate for his/her head size, the appropriate sized gel pack is inserted, and the wrap 10 then positioned about the user's head. Preferably, the appropriate size gel pack is sold with each size of the wrap 10.

From the foregoing description, one skilled in the art can easily ascertain the essential characteristics of this invention and, without departing from the spirit and scope thereof, can make various changes and modifications of the invention to adapt it to various usages and conditions.

It is to be understood that the present invention is not limited to the sole embodiment described above, but encompasses any and all embodiments within the scope of the following claims:

I claim:

1. A thermal wrap comprising:
a pair of opposing side portions, each of said side portions having inner and outer sheets, said inner sheet connected to an interior facing surface of a pair of connecting straps and said outer sheet connected to an exterior facing surface of said pair of connecting straps, the connecting straps connecting the top ends of the side portions;
a chin strap connected to lower edges of said side portions;
a single sheet of material connected proximate the lower edges of the side portions, and also secured lengthwise thereto with an open top and thereby defining a pocket sized to receive standard sized gel packs.

2. The wrap of claim 1 wherein each of said connecting straps have a first segment having adjustable attachment means connected thereto, and a second segment having means for securing to one of said side pockets, said second segment attachable to an opposing side of said attachment means.

3. The wrap of claim 2 wherein said adjustable attachment means is a buckle.

4. The wrap of claim 1 wherein said chin strap is at least half the width of said side pockets.

5. The wrap of claim 1 wherein said chin strap is formed of elasticated material.
